(19) United States
(12) Patent Application Publication
(10) Pub. No.: US 2007/0229080 A1
(43) Pub. Date: Oct. 4, 2007

(54) Electro-Optical Magnetic Resonance Transducer

(75) Inventors: Steffen Weiss, Hamburg (DE); Tim Nielsen, Hamburg (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/568,125

(22) PCT Filed: Mar. 30, 2005
(86) PCT No.: PCT/IB05/51069

§ 371 Date: Oct. 20, 2006

Related U.S. Application Data
(60) Provisional application No. 60/565,375, filed on Apr. 26, 2004.

(51) Int. Cl. G01R 33/465; A61B 5/05

(52) U.S. Cl. 324/322; 600/423

(57) ABSTRACT

A receive coil (46, 46′, 46″) receiving a magnetic resonance signal includes an optical resonant cavity (82, 82′, 82″) having an electro-optically active medium (90, 90′, 90″). A radio frequency antenna (80, 80′, 80″) is coupled with the electro-optically active medium such that reception of the magnetic resonance signal by the radio frequency antenna modulates an optical characteristic of the optical resonant cavity. An interventional instrument (48) is adapted for partial insertion into an associated imaging subject (16). The radio frequency antenna and the optical resonant cavity are disposed on a portion of the interventional instrument that is inserted into the associated imaging subject. At least one optical fiber (52, 54) is optically coupled with the optical resonant cavity. An end of the at least one optical fiber extends outside of the associated imaging subject when the interventional instrument is partially inserted.

The following relates to the magnetic resonance arts. It finds particular application in magnetic resonance imaging using a local receive coil disposed on a catheter or other interventional instrument that is partially inserted into an imaging subject, and will be described with particular reference thereto. However, it also finds application in sensitive electro-optical transducers for converting weak electrical signals into optical signals for magnetic resonance and other applications.

Radio frequency receive coils are advantageously disposed near or inside of a magnetic resonance imaging subject to improve coupling with the magnetic resonance signal produced by a region of interest of that subject. Surface receive coils, for example, are disposed outside the imaging subject near or in contact with the exterior of the region of interest. For still closer coupling with an internal region of interest, the receive coil can be disposed on a catheter, biopsy needle, or other interventional instrument. The portion of the interventional instrument on which the receive coil is disposed is inserted into the imaging subject such that the receive coil is disposed close to, or inside of, the region of interest. Such an arrangement can also be used to provide magnetic resonance imaging visualizations for guiding a surgeon who is performing an interventional procedure using the interventional instrument.

However, problems arise in placing the receive coil close to or inside of the imaging subject. One problem relates to the use of conductive wires to transmit the received magnetic resonance signal to a radio frequency receiver or other signal processing equipment. The conductive wires can inductively heat in the magnetic resonance imaging environment, producing a safety hazard. The conductive wire may also distort radio frequency fields in the imaging subject, producing imaging artifacts.

One approach to addressing these difficulties is to employ a wireless receive coil, in which a transmitter disposed on or near the receive coil shifts the received magnetic resonance signal to another frequency range and transmits the frequency-shifted signal to an external receiver. This approach has disadvantage in that the hardware for performing the heterodyning of the magnetic resonance signal must be disposed on the receive coil. This is difficult in the case of surface coils, and more difficult in the case of a receive coil disposed on a catheter or other interventional instrument. Moreover, the heterodyning circuitry generally requires electrical power typically supplied by electrical power conductors, which again may introduce difficulties such as conductor heating or introduction of imaging artifacts.

Another approach is described in the International Application of inventors Konings and Weiss (WO 02/086526 A1, published Oct. 31, 2002). The disclosed approach employs optical fibers to input linearly polarized light to the receive coil. An electro-optical modulator connected with the coil has crossed polarizers with an electro-optically active material disposed therebetween. The electro-optically active material produces a polarization rotation. The two polarizers are crossed to extinguish the light in the absence of an applied electric field. The radio frequency antenna of the receive coil imposes an oscillating electric field on the electro-optically active material which rotates the polarization, permitting some light to pass through the crossed polarizers. This approach has certain sensitivity difficulties. The crossed polarizers should be arranged to accurately null the light transmission in the absence of an applied electric field, so that the slight change in optical rotation is detectable with adequate dynamic range. This nulling is complicated by Faraday rotation produced by the strong magnetic fields and magnetic field gradients present in the magnetic resonance imaging environment. The amount of Faraday rotation changes with changes in the magnetic field, such as are produced by magnetic field gradient coils. The Faraday rotation competes with and can obscure the electro-optical rotation.

The present invention contemplates an improved apparatus and method that overcomes the aforementioned limitations and others.

According to one aspect, a receive coil for magnetic resonance imaging is disclosed. An optical resonant cavity includes an electro-optically active medium. A radio frequency antenna is coupled with the electro-optically active medium such that reception of a magnetic resonance signal by the radio frequency antenna modulates an optical characteristic of the optical resonant cavity. At least one optical fiber is optically coupled with the optical resonant cavity.

According to another aspect, a method is provided for detecting magnetic resonance. Light is optically coupled with an optical resonant cavity. The optically coupled light is modulated using a radio frequency antenna to produce modulated light. The modulating includes electro-optically modulating an electro-optically active medium of the optical resonant cavity responsive to reception of a magnetic resonance signal by the radio frequency antenna. An optical characteristic of the optical resonant cavity is measured based on the modulated light.

According to yet another aspect, a magnetic resonance imaging scanner is disclosed. A magnet generates a temporally constant magnetic field in an imaging region. One or more magnetic field gradient coils superimpose selected magnetic field gradients on the temporally constant magnetic field in the imaging region. A radio frequency transmitter injects a magnetic resonance excitation signal into the imaging region. A receive coil is provided. An optical resonant cavity includes an electro-optically active medium. A radio frequency antenna is coupled with the electro-optically active medium such that reception of a magnetic resonance signal by the radio frequency antenna modulates an optical characteristic of the optical resonant cavity. An interventional instrument is adapted for partial insertion into an associated imaging subject. The radio frequency antenna and the optical resonant cavity are disposed on a portion of the interventional instrument that is inserted into the associated imaging subject. At least one optical fiber is optically coupled with the optical resonant cavity. An end of the at least one optical fiber extends outside of the associated imaging subject when the interventional instrument is partially inserted.

One advantage resides in providing a safe magnetic resonance receive coil that does not include electrical inputs or outputs.

Another advantage resides in improved sensitivity to the magnetic resonance signal. The sensitivity can be improved by a factor of about 10⁴ to 10⁵ as compared with the polarization rotation-based electro-optic modulator of WO 02/086526 A1 which was discussed in the Background of the Invention.

Yet another advantage resides in reduced or eliminated sensitivity to magnetic field-induced Faraday rotation effects.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

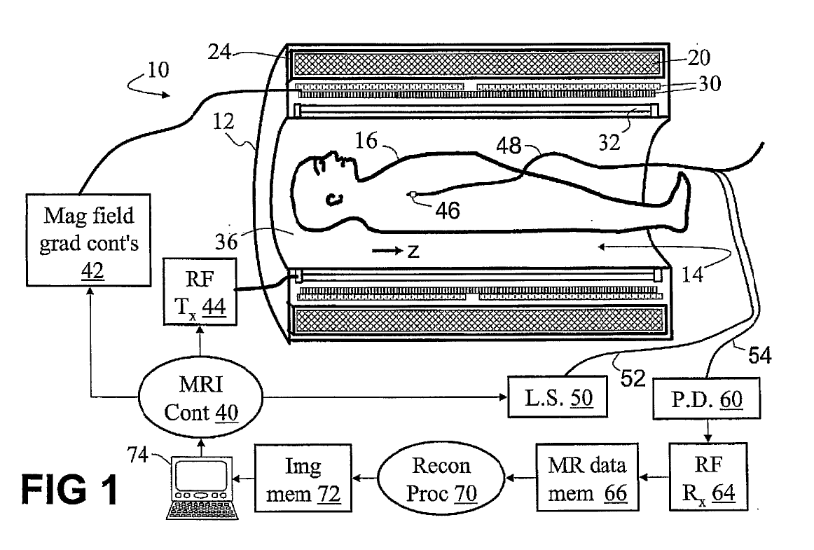

FIG. 1 diagrammatically shows a magnetic resonance imaging system including a magnetic resonance receive coil disposed on a catheter.

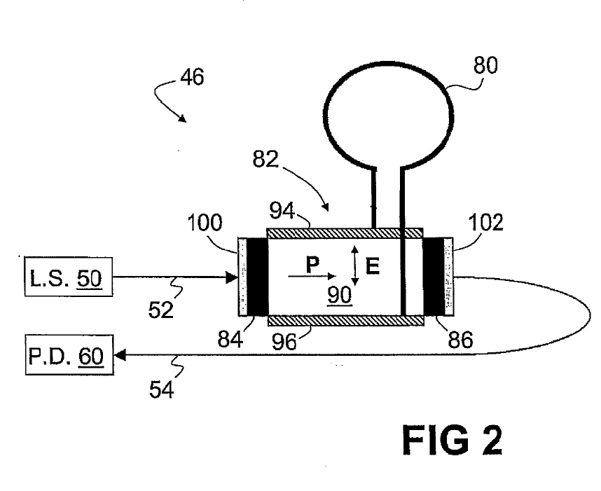

FIG. 2 diagrammatically shows an embodiment of the magnetic resonance receive coil of FIG. 1.

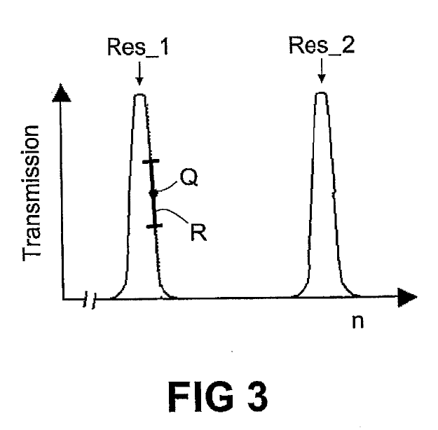

FIG. 3 diagrammatically plots a transmission characteristic of the optical resonant cavity of the magnetic resonance receive coil.

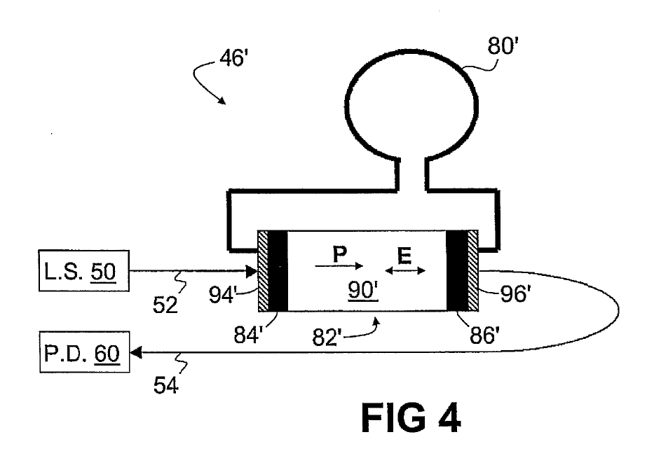

FIG. 4 diagrammatically shows another embodiment of the magnetic resonance receive coil of FIG. 1.

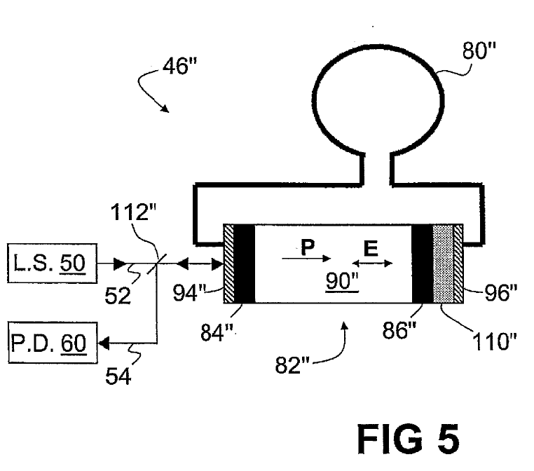

FIG. 5 diagrammatically shows yet another embodiment of the magnetic resonance receive coil of FIG. 1.

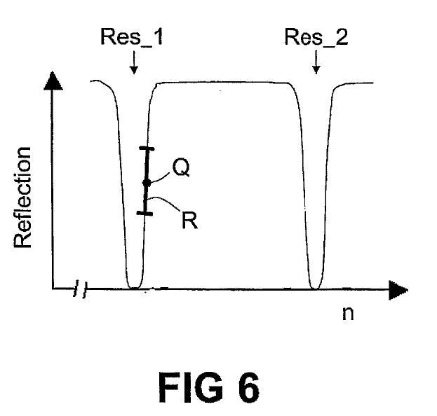

FIG. 6 diagrammatically plots a reflection characteristic of the optical resonant cavity of the magnetic resonance receive coil of FIG. 5.

With reference to FIG. 1, a magnetic resonance imaging scanner 10 includes a housing 12 defining a generally cylindrical scanner bore 14 inside of which an associated imaging subject 16 is disposed. Main magnetic field coils 20 are disposed inside the housing 12, and produce a temporally constant B₀ magnetic field directed generally along a direction, designated the z-direction in FIG. 1, which is substantially parallel to a central axis of the scanner bore 14.

The housing 12 also houses or supports magnetic field gradient-generating structures, such as magnetic field gradient coils 30, for selectively producing magnetic field gradients parallel to the z-direction, transverse to the z-direction, or along other selected directions. The housing 12 further houses or supports a radio frequency body coil 32 for selectively exciting magnetic resonances. Specifically, the radio frequency body coil 32 produces a radio frequency B, magnetic field transverse to the temporally constant B₀ magnetic field. The radio frequency B, magnetic field is generated at the Larmor frequency for exciting a nuclear magnetic resonance. In the illustrated embodiment, the coil 32 is a whole body birdcage coil; however, a local coil, a whole-body TEM coil, or other radio frequency coil can be used for exciting magnetic resonance in the subject 16. The housing 12 typically includes a cosmetic inner liner 36 inside the birdcage coil 32 defining the scanner bore 14.

During imaging, the main magnetic field coils 20 produce the temporally constant B₀ magnetic field parallel to the z-direction in the bore 14. A magnetic resonance imaging controller 40 operates magnetic field gradient controllers 42 to selectively energize the magnetic field gradient coils 30, and operates a radio frequency transmitter 44 coupled to the radio frequency coil 32 to selectively energize the radio frequency coil 32. By selectively operating the magnetic field gradient coils 30 and the radio frequency coil 32, magnetic resonance is generated and spatially encoded in at least a portion of a region of interest of the imaging subject 16. By applying selected magnetic field gradients via the gradient coils 30, a selected k-space trajectory is traversed during acquisition of magnetic resonance signals, such as a Cartesian trajectory, a plurality of radial trajectories, or a spiral trajectory.

The described magnetic resonance imaging scanner is an example. The magnetic field receive coils described herein can be employed with substantially any type of magnetic resonance imaging scanner, such as an open magnet scanner, a vertical magnet scanner, or so forth. Moreover, the magnetic field receive coils described herein can be employed in magnetic resonance procedures other than imaging, such as in magnetic resonance spectroscopy.

During imaging data acquisition, a radio frequency receive coil 46 disposed on a catheter 48 inserted into the imaging subject 16, or another coil such as a surface receive coil, is used to acquire magnetic resonance samples. The radio frequency receive coil 46 is optically coupled for extracting the magnetic resonance signal. Specifically, a light source 50, such as a laser, a lamp, or the like, delivers input light into an input optical fiber 52 that passes through a lumen of the catheter 48 to couple with the receive coil 46. The receive coil produces electro-optical modulation of the input light at the magnetic resonance frequency to produce a modulated output light. An output optical fiber 56 passes through a lumen of the catheter 48 and couples with a photodetector 60, such as a photodiode, a photomultiplier tube, a spectrometer, or the like. The output of the photodetector 60 corresponds to the magnetic resonance signal. The light source 50 is optionally controlled by the magnetic resonance imaging controller 40 to turn the light source on and off, to control the wavelength to match an operating frequency of the electro-optical modulator, or the like.

In some embodiments, the fibers 52, 56 are the same fiber, and an optical coupler/splitter or other optical component or combination of components is used to couple the input light to the fiber and to extract modulated light from the fiber. Still further, in some embodiments the catheter 48 is replaced by a biopsy needle or other interventional instrument. Optionally, the catheter 48 or other interventional instrument also includes hardware for performing an interventional medical procedure, such as hardware for performing balloon angioplasty, and the magnetic resonance imaging employing the radio frequency receive coil 46 is used for image-based surgical guidance. As yet another option, the receive coil can be inserted into the patient and the insertion/removal tools removed to a remote location during imaging.

In some embodiments, the photodetector 60 produces a radio frequency signal corresponding to an amplitude or intensity of the magnetic resonance signal. The radio frequency electrical signal is demodulated by a radio frequency receiver 64, which is typically a digital receiver although an analog receiver can also be used, to produce magnetic resonance data that are stored in a magnetic resonance data memory 66. The magnetic resonance data are reconstructed by a reconstruction processor 70 into a reconstructed image. In the case of k-space sampling data, a Fourier transform-based reconstruction algorithm can be employed. Other reconstruction algorithms, such as a filtered backprojection-based reconstruction, can also be used depending upon the format of the acquired magnetic resonance imaging data. The reconstructed image generated by the reconstruction processor 70 is stored in an image memory 72, and can be displayed on a user interface 74, stored in non-volatile memory, transmitted over a local intranet or the Internet, viewed, stored, manipulated, or so forth. The user interface 74 can also enable a radiologist, technician, or other operator of the magnetic resonance imaging scanner 10 to communicate with the magnetic resonance imaging controller 40 to select, modify, and execute magnetic resonance imaging sequences.

With reference to FIG. 2, in one embodiment the receive coil 46 includes a radio frequency receive antenna 80 tuned to detect the magnetic resonance. The antenna 80 is coupled with an optical resonant cavity 82 including mirrors 84, 86 that define a length of the resonant cavity. The optical resonant cavity 82 includes an electro-optically active medium 90 disposed between the mirrors 84, 86.

With continuing reference to FIG. 2 and with brief reference to FIG. 3, the optical resonant cavity 82 including the mirrors 84, 86 and the electro-optically active medium 90 define a Fabry-Perot resonator whose resonance frequencies depend upon the refractive index (“n”) of the electro-optically active medium 90. For example, as plotted in FIG. 3, a transmission characteristic of the optical resonant cavity 82 for a particular wavelength varies as a function of the refractive index of the electro-optically active medium 90 seen by the propagating light. Certain refractive index values correspond to a resonance condition for that particular wavelength, and at such a resonance condition the transmission of the optical resonant cavity 82 becomes large. In some embodiments, the transmission approaches unity at resonance. In FIG. 3, two such resonance conditions are indicated as “Res_{—}1” and “Res_{—}2”. On the other hand, for refractive index values substantially different from a resonance condition, the transmission of the optical resonant cavity 82 becomes small. In some embodiments, the transmission approaches zero away, from a resonance condition.

With continuing reference to FIG. 2, the light source 50 injects light into the optical resonant cavity 82. The injected light couples into the optical resonant cavity 82 and passes through the electro-optically active medium 90 in a propagation direction P. Preferably, the electro-optically active medium 90 is transparent or substantially light transmissive for light produced by the light source 50. The antenna 80 is coupled with the electro-optically active medium 90 through electrodes 94, 96 arranged relative to the optical resonant cavity 82 so as to produce an electric field E directed transverse to the propagation direction P of the light. The electric field E modifies the refractive index of the electro-optically active medium 90. The injected light is preferably polarized in the direction of the electric field E by an input polarizer 100 so as to maximize the refractive index modulation seen by the propagating light.

With continuing reference to FIG. 2 and with returning reference to FIG. 3, in one embodiment the light source 50 produces a substantially monochromatic light for which the transmission characteristic of the optical resonant cavity 82 is plotted in FIG. 3. For example, the light source 50 can be a monochromatic laser, or it can be a broad-band lamp with suitable narrow band-pass filtering to derive substantially monochromatic light. The optical resonant cavity 82 is tuned to a quiescent operating point Q in the absence of an electric field E applied by the antenna 80. The quiescent operating point Q corresponds to a slightly off-resonance condition in which the transmissivity slope as a function of refractive index is large.

When the antenna 80 receives a magnetic resonance signal, it develops an induced oscillating voltage that in turn produces the electric field E in the electro-optically active medium 90 which oscillates at the magnetic resonance frequency. The oscillating electric field E produces a corresponding oscillating refractive index value of the electro-optically active medium 90 which causes the transmission characteristic of the optical resonant cavity 82 to oscillate about the quiescent operating point Q over a range R indicated in FIG. 3.

In some embodiments, the light source 50 is monochromatic, and the photodetector 60 measures the amplitude or intensity of the light produced by the light source 50 that is transmitted through and modulated by the optical resonant cavity 82. This amplitude or intensity has an oscillating variation corresponding to the oscillating electric field E caused by the magnetic resonance signal. The background d.c. output of the photodetector 60 is suitably removed by band-pass or high-pass filtering, leaving a radio frequency signal corresponding to the magnetic resonance signal.

In other embodiments, the light source 50 is a relatively broad-band light source producing polychromatic light over a selected wavelength range. The optical resonant cavity 82 acts as a narrow-band filter passing the resonant wavelength. As the oscillating electric field E oscillates the refractive index value of the electro-optically active medium 90, the resonant frequency correspondingly oscillates. In these embodiments, the photodetector 60 is a spectrometer, and the wavelength shift of the resonance condition caused by the applied electric field E and measured by the spectrometer corresponds to the magnetic resonance signal intensity.

While the described embodiments operate based on electro-optical changes in refractive index, it is also contemplated to modulate the light by electrically changing the absorption properties of the active medium via the electro-absorption or Franz-Keldysh effect. For active media exhibiting a substantial electro-absorption, it may be possible to omit the optical resonant cavity 82.

The radio frequency receive coil 46 operates inside the bore 14 of the magnetic resonance imaging scanner 10. This environment includes large magnetic fields and magnetic field gradients, which can induce Faraday rotation in light propagating through the electro-optically active medium 90, and perhaps also in the light passing through the optical fiber 52. In some embodiments, the light source 50 produces circularly polarized light, which is unaffected by any Faraday rotation which occurs during light transmission through the input optical fiber 52. The input linear polarizer 100 is preferably disposed close to the optical resonant cavity 82 to further reduce extraneous Faraday rotation effects. Optionally, a second, output polarizer 102 disposed at the output of the optical resonant cavity 82 substantially removes depolarized or scattered light to improve the signal-to-noise ratio.

The radio frequency receive coil 46 can be constructed in various ways. The antenna 80 is suitably a printed circuit, conductive coil, or other receive antenna such as are typically used to instrument catheters, biopsy needles, other interventional instruments, or other local coils. In one embodiment the optical resonant cavity 82 is an optical fiber portion made of quartz, KH₂PO₄ (KDP), beta barium borate (BBO), or another electro-optically active material, with the electrodes 94, 96 evaporated on the outside of the optical fiber. These KDP, BBO, and certain other electro-optical materials are suitable for operation in the visible or near-infrared wavelength range.

In another embodiment, the optical resonant cavity 82 is embodied as a thin film or chip of KDP, BBO, or another electro-optically active material with the electrodes 94, 96 and mirrors 84, 86 evaporated or otherwise deposited thereon. In yet another embodiment, a GaAs/AlGaAs resonant cavity is employed, in which the mirrors 84, 86 are distributed Bragg reflectors (DBR's) defined by stacks of alternating GaAs and AlGaAs layers or by stacks of alternating layers of AlGaAs of different aluminum compositions. GaAs-based resonant cavities are typically tuned to an infra-red wavelength or a visible wavelength preferably in the red range. Other heteroepitaxial semiconductor-based resonant cavity structures can also be employed, such as, for example, a group III-phosphide resonant cavity structure.

A reduced separation of the electrodes 94, 96 such as is provided by a thin film advantageously produces large electric fields E. However, the separation of the electrodes 94, 96 should be large enough so that the optical resonant cavity 82 has good light coupling and throughput, at least commensurate with the transmission fibers 52, 54. Those skilled in the art can readily select dimensions that advantageously balance these considerations in specific embodiments.

With reference to FIG. 4, another embodiment of the radio frequency receive coil 46′ includes a radio frequency antenna 80′ coupled with a resonant cavity 82′ having a cavity length defined by mirrors 84′, 86′ and including an electro-optically active medium 90′ disposed between the mirrors 84′, 86′. These elements are similar to the corresponding elements of the receive coil 46 of FIG. 2, and light propagates in a propagation direction P in the optical resonant cavity as in the receive coil 46.

The receive coil 46′ differs from the receive coil 46 in that the receive coil 46′ includes electrodes 94′, 96′ disposed on the same sides of the optical resonant cavity 46′ as the mirrors 84′, 86′ to produce an electric field E directed along the propagation direction P, rather than transverse to the propagation direction P as in the receive coil 46. While separate mirrors 84′, 86′ and electrodes 94′, 96′ are illustrated, in some embodiments the mirrors may also serve as electrodes. If the electrodes 94′, 96′ are distinct from the mirrors 84′, 86′, then the electrodes 94′, 96′ should be substantially transparent to light produced by the light source 50. For example, thin indium tin oxide (ITO) electrodes can be employed.

Since in the optical resonant cavity 82′ the electric field E is parallel with and directed along the propagation direction P, modulation of the refractive index of the electro-optically active medium 90′ produced by the magnetic resonance signal causes the transmission characteristic of the optical resonant cavity 82′ to oscillate for any polarization of the propagating light. Hence, no polarizer is needed, and moreover the radio frequency receive coil 46′ is insensitive to Faraday rotation. Because the electric field E is produced by applying the induced voltage of the coil 80′ across the length of the optical resonant cavity 82′, the cavity length is selected based on competing considerations of (i) providing a relatively long cavity so that the cavity has a large quality factor and reduced capacitance, and (ii) providing a relatively short cavity so that the oscillating electric field E has a large enough amplitude or intensity to produce a substantial electro-optic effect. A cavity length of about 3-5 millimeters is suitable for some embodiments operating in the visible or near-infrared.

The receive coil 46′ is operated similarly to the operation of the receive coil 46. In one embodiment, the light source 50 is monochromatic, and the optical resonant cavity 82′ has a quiescent operating point selected to provide a large transmission slope as a function of refractive index. Modulation produced by the magnetic resonance signal received by the antenna 80′ modulates the refractive index of the electro-optically active medium 90′ which in turn modulates the intensity of the transmitted monochromatic light. The photodetector 60 detects the amplitude or intensity of the modulated transmitted monochromatic light, and the radio frequency component of the output of the photodetector 60 is recovered by suitable filtering. Alternatively, the light source 50 is polychromatic, the photodetector 60 is a spectrometer, and the magnetic resonance signal is determined based on the wavelength shift produced by shifting of the resonance wavelength of the optical resonant cavity 82′ caused by the oscillating electric field E.

In both receive coils 46, 46′, an optical transmission characteristic of the optical resonant cavity 82, 82′ is measured to determine the magnetic resonance signal.

With reference to FIG. 5, still yet other embodiments of the radio frequency receive coil 46″ employ a reflection geometry. The receive coil 46″ includes a radio frequency antenna 80″ coupled with a resonant cavity 82″ having a cavity length defined by mirrors 84″, 86″ and including an electro-optically active medium, 90″ disposed between the mirrors 84″, 86″. Electrodes 94″, 96″ are disposed on the same sides of the optical resonant cavity 82″ as the mirrors 84″, 86″ to produce an electric field E directed along the light propagation direction P. These elements of the receive coil 46″ are similar to the corresponding elements of the receive coil 46′ of FIG. 4.

The receive coil 46″ differs from the receive coil 46′ in that the optical resonant cavity 82″ includes a light-absorbing region 110″ that absorbs light produced by the light source 50. Moreover, the optical geometry is different. Rather than measuring a transmission characteristic, the receive coil 46″ includes an optical coupler/decoupler, such as a beam splitter 112″. Light is coupled from the light source 50 to the optical resonant cavity 82″ via an optical fiber. Light reflected from the optical resonant cavity 82″ reflects back into the optical fiber and propagates back to the beam splitter 112″, which diverts at least a portion of the reflected light to the photodetector 60.

With continuing reference to FIG. 5 and with further reference to FIG. 6, in one embodiment the light source 50 produces a substantially monochromatic light for which the reflection characteristic of the optical resonant cavity 82″ is plotted in FIG. 6. When the refractive index value of the electro-optically active medium 90″ is such that the light is away from resonance, then the optical resonant cavity 82″ is not optically transmissive, but rather is optically reflective. Thus, the reflection characteristic is large off resonance. However, when the refractive index value of the electro-optically active medium 90″ is such that the light is at a resonance condition, the light enters the resonant cavity 82″ and passes through the cavity. However, the light transmitted through the optical resonant cavity 82″ is substantially absorbed by the light-absorbing region 110″. Hence, when the refractive index value tunes the cavity to resonate with the monochromatic light, the light is substantially absorbed in light-absorbing region 110″ and the reflection characteristic decreases. In some embodiments, the reflectivity is about unity off resonance, and decreases to about zero at a resonant condition. In FIG. 6, two illustrated resonant conditions are labeled “Res_{—}1” and Res_{—}2″.

When the antenna 80″ receives a magnetic resonance signal, it develops an induced radio frequency voltage producing the oscillating electric field E, which in turn produces an oscillating refractive index value of the electro-optically active medium 90 which causes the reflection characteristic of the optical resonant cavity 82 to oscillate about a quiescent operating point Q over a range R indicated in FIG. 6. The quiescent operating point Q is selected to correspond to a region in which the slope of the reflectivity as a function of refractive index is large. The photodetector 60 measures the amplitude or intensity of the light produced by the light source 50 that is reflected by the optical resonant cavity 82. This amplitude or intensity has an oscillating variation corresponding to the oscillating electric field E caused by the magnetic resonance signal. The background d.c. output of the photodetector 60 is suitably removed by band-pass or high-pass filtering, leaving a radio frequency signal corresponding to the magnetic resonance signal.

The described magnetic resonance receive coils 46, 46′, 46″ are suitable for mounting on or inside of an interventional instrument, such as the example catheter 48. A portion of the interventional instrument is inserted into the imaging subject 16 to place the magnetic resonance receive coil 46, 46′, 46″ close to the region of interest. Optionally, the imaging is performed during an interventional surgical procedure to provide the surgeon with visual guidance while performing the surgical procedure.

It will be appreciated that the magnetic resonance receive coils 46, 46′, 46″ can be used in other settings besides on or in an interventional instrument. For example, those skilled in the art can readily construct a wireless surface receive coil, an array of wireless receive coils, or the like which employ the electro-optical modulation techniques of the described magnetic resonance receive coils 46, 46′, 46″.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A receive coil for detecting magnetic resonance signals, the receive coil comprising:
an optical resonant cavity including an electro-optically active medium;
a radio frequency antenna coupled with the electro-optically active medium such that reception of a magnetic resonance signal by the radio frequency antenna modulates an optical characteristic of the optical resonant cavity; and
at least one optical fiber optically coupled with the optical resonant cavity.

2. The receive coil as set forth in claim 1, further comprising:
an interventional instrument adapted for partial insertion into an associated imaging subject, the radio frequency antenna and the optical resonant cavity being disposed on or in a portion of the interventional instrument that is inserted into the associated imaging subject, an end of the at least one optical fiber extending outside of the associated imaging subject.

3. The receive coil as set forth in claim 2, further comprising:
electrodes coupled with the radio frequency antenna, the electrodes arranged to produce an electric field in the electro-optically active medium directed transverse to a direction of light transmission through the optically resonant cavity.

4. The receive coil as set forth in claim 3, further comprising:
a light source producing circularly polarized light inputted to the at least one optical fiber; and
a polarizer disposed between the at least one optical fiber and the optical resonant cavity, the polarizer converting the circularly polarized light into linearly polarized light.

5. The receive coil as set forth in claim 2, further comprising:
electrodes coupled with the radio frequency antenna, the electrodes arranged to produce an electric field in the electro-optically active medium directed parallel with a direction of light propagation in the optically resonant cavity.

6. The receive coil as set forth in claim 2, further comprising:
a substantially monochromatic light source producing substantially monochromatic light inputted to the at least one optical fiber; and
a photodetector arranged to detect an amplitude or intensity of the inputted substantially monochromatic light after interacting with the optical resonant cavity.

7. The receive coil as set forth in claim 2, further comprising:
a light source producing polychromatic light spanning a band of wavelengths inputted to the at least one optical fiber; and
a spectrometer arranged to measure a spectrum of the inputted light after interacting with the optical resonant cavity.

8. The receive coil as set forth in claim 5, wherein the at least one optical fiber includes:
an input optical fiber that injects light into an input side of the optical resonant cavity; and
an output optical fiber that receives light passing out of an output side of the optical resonant cavity opposite the input side.

9. The receive coil as set forth in claim 5, further comprising:
a light-absorbing region disposed behind the optical resonant cavity that absorbs light transmitted through the optical resonant cavity.

10. The receive coil as set forth in claim 5, wherein the electrodes additionally serve as minors of the optical resonant cavity.

11. The receive coil as set forth in claim 1, further comprising:
electrodes coupled with the radio frequency antenna, the electrodes arranged to produce an electric field in the electro-optically active medium directed transverse to a direction of light transmission through the optical resonant cavity; and
a polarizer disposed between the at least one optical fiber and the optical resonant cavity, the polarizer converting the circularly polarized light into linearly polarized light.

12. The receive coil as set forth in claim 1, further comprising:
electrodes coupled with the radio frequency antenna, the electrodes arranged to produce an electric field in the electro-optically active medium directed parallel with a direction of light transmission through in the optical resonant cavity; =p1 an input optical fiber that injects light into an input side of the optical resonant cavity; and
an output optical fiber that receives light passing out of an output side of the optically resonant cavity opposite the input side.

13. The receive coil as set forth in claim 1, further comprising:
electrodes coupled with the radio frequency antenna, the electrodes arranged to produce an electric field in the electro-optically active medium directed parallel with a direction of light propagation in the optical resonant cavity;
an absorption region disposed behind the optically resonant cavity that absorbs light transmitted through the optical resonant cavity; and
an optical coupler coupling an input light into the at least one fiber and extracting reflected light reflected from the optically resonant cavity from the at least one fiber.

14. The receive coil as set forth in claim 1, wherein the modulated optical characteristic is selected from a group consisting of refractive index and absorption.

15. A method for detecting a magnetic resonance signal, the method comprising:
optically coupling light with an optical resonant cavity;
modulating the optically coupled light using a radio frequency antenna to produce modulated light, the modulating including electro-optically modulating an electro-optically active medium of the optical resonant cavity responsive to reception of the magnetic resonance signal by the radio frequency antenna; and
measuring an optical characteristic of the optical resonant cavity based on the modulated light.

16. The method as set forth in claim 15, wherein the radio frequency antenna and the optical resonant cavity are disposed on or in an interventional instrument, the method further comprising:
inserting a portion of the interventional instrument having the radio frequency antenna and the optical resonant cavity disposed thereon or therein into an associated imaging subject, an optical fiber extending outside the imaging subject when the interventional instrument is inserted providing optical access to the optical resonant cavity.

17. The method as set forth in claim 15, wherein the modulating comprises:
producing an electric field transverse to a direction of light propagation in the optical resonant cavity.

18. The method as set forth in claim 17, wherein the optical coupling of light comprises:
linearly polarizing light, the linearly polarized light being optically coupled with the optical resonant cavity.

19. The method as set forth in claim 15, wherein the modulating comprises:
producing an electric field along a direction of light propagation in the optical resonant cavity.

20. The method as set forth in claim 19, wherein the measuring of an optical characteristic of the optical resonant cavity comprises:
measuring an optical transmission characteristic of the optical resonant cavity.

21. The method as set forth in claim 19, wherein the measuring of an optical characteristic of the optical resonant cavity comprises:
absorbing at least a portion of light behind the optical resonant cavity; and
measuring an optical reflection characteristic of the optical resonant cavity.

22. A method for detecting a magnetic resonance signal, the method comprising:
optically coupling light with an active medium exhibiting electro-absorption;
modulating the optically coupled light using a radio frequency antenna to produce modulated light, the modulating including electro-absorption modulation of the active medium responsive to reception of the magnetic resonance signal by the radio frequency antenna; and
measuring an optical characteristic of the active medium based on the modulated light.

23. A magnetic resonance imaging scanner comprising:
a magnet generating a temporally constant magnetic field in an imaging region;
one or more magnetic field gradient coils superimposing selected magnetic field gradients on the temporally constant magnetic field in the imaging region;
a radio frequency transmitter injecting a magnetic resonance excitation signal into the imaging region; and
a receive coil comprising:
an optical resonant cavity including ant electro-optically active medium,
a radio frequency antenna coupled with the electro-optically active medium such that reception of a magnetic resonance signal by the radio frequency antenna modulates an optical characteristic of the optical resonant cavity, =p2 an interventional instrument adapted for partial insertion into an associated imaging subject, the radio frequency antenna and the optical resonant cavity being disposed on a portion of the interventional instrument that is inserted into the associated imaging subject, and
at least one optical fiber optically coupling with the optical resonant cavity, an end of the at least one optical fiber extending outside of the associated imaging subject when the interventional instrument is partially inserted.

24. A receive coil for converting electromagnetic signals to optical signals, the receive coil comprising:
an optical resonant cavity including an electro-optically active medium;
a radio frequency antenna coupled with the electro-optically active medium such that reception of an electromagnetic signal by the radio frequency antenna modulates an optical characteristic of the optical resonant cavity; and
at least one optical fiber optically coupled with the optical resonant cavity.
